(19) United States
(12) Patent Application Publication
(10) Pub. No.: US 2025/0037824 A1
(43) Pub. Date: Jan. 30, 2025

(54) SUMMARIZATION INTELLIGENT PIPELINE TOOL SYSTEMS AND METHODS

(71) Applicant: Allstate India Private Limited, Bangalore (IN)

(72) Inventors: Balaji Jagan, Bangalore (IN); Prasenjeet Roy, Bangalore (IN)

(21) Appl. No.: 18/785,035

(22) Filed: Jul. 26, 2024

(30) Foreign Application Priority Data
Jul. 27, 2023 (IN) 202341050652

(51) Int. Cl. G16H 15/00; G16H 10/60

(52) U.S. Cl. CPC G16H 15/00; G16H 10/60

(57) ABSTRACT

Industry report summarization intelligent pipeline tool systems and methods includes one or more processors, one or more memory components communicatively coupled to the one or more processors, and machine-readable instructions. The machine-readable instructions cause the system to perform methods as described herein to automatically generate a domain-independent industry report with in-domain ontology for the industry.

### CROSS-REFERENCE TO RELATED APPLICATIONS

The present application claims priority to Indian Provisional App. No. 202341050652, filed on Jul. 27, 2023, and entitled “SUMMARIZATION INTELLIGENT PIPELINE TOOL SYSTEMS AND METHODS,” the entirety of which is incorporated by reference herein.

### TECHNICAL FIELD

The present specification generally relates to summarization intelligent pipeline tool systems and methods, and more particularly, to systems and methods for a medical report summarization intelligent pipeline tool.

### BACKGROUND

With the increasing use of industry reporting generated very long reports often in the hundreds or thousands of pages associated with an industry client, there is a growing need for efficient, accurate, and timely report summarization.

### SUMMARY

According to the subject matter of the present disclosure, a system may include one or more processors, one or more memory components communicatively coupled to the one or more processors, and one or more machine readable instructions stored in the one or more memory components. The machine-readable instructions may cause the system to perform at least the following when executed by the one or more processors: one or more logical processes as described herein.

According to an embodiment, a system for intelligent pipeline summarization for a medical report of a patient comprises a processor and a memory storing computer-executable instructions that, when executed by the processor, cause the system to: receive one or more medical records associated with the patient for summarization for the medical report of the patient, and extract one or more potential headings from one or more phrases of a medical record text of the one or more medical records. The computer-executable instructions, when executed by the processor, further may cause the system to: compare the one or more potential headings with one or more keyword headings of a heading seed set to generate, upon a match, one or more segmentation headings; segment, via a text segmentation algorithm of an artificial intelligence model, one or more sections of the medical record text of the one or more medical records as a set of segmented texts based on the one or more segmentation headings, generate a semantic graph representation based on at least the set of segmented texts that is representative of a semantic relationship as a medical semantic type between at least a plurality of nodes, extract one or more medical entities based on an annotated medical domain ontology from the generated semantic graph representation, prune a set of irrelevant information based on the annotated medical domain ontology applied to the generated semantic graph representation, and generate a medically relevant summary for the medical report based on the extracted one or more medical entities and excluding the pruned set of irrelevant information based on the annotated medical domain ontology.

According to yet another embodiment, a system for intelligent pipeline summarization for a medical report of a patient comprises a processor and a memory storing computer-executable instructions. When executed by the processor, the computer-executable instructions may cause the system to receive one or more medical records associated with the patient for summarization for the medical report of the patient, and extract one or more potential headings from one or more phrases of a medical record text of the one or more medical records. The computer-executable instructions, when executed by the processor, further may cause the system to: compare the one or more potential headings with one or more keyword headings of a heading seed set to generate, upon a match, one or more segmentation headings, segment, via a text segmentation algorithm of an artificial intelligence model, one or more sections of the medical record text of the one or more medical records as a set of segmented texts based on the one or more segmentation headings, and classify, via a text classification algorithm of the artificial intelligence model, the set of segmented texts with one or more pre-defined medical tags to generate one or more groups of classified text, wherein the one or more groups of classified text comprise a number of predefined categories. The computer-executable instructions, when executed by the processor, further may cause the system to: generate a semantic graph representation based on at least the one or more groups of classified text that is representative of a semantic relationship as a medical semantic type between at least a plurality of nodes, extract one or more medical entities based on an annotated medical domain ontology from the generated semantic graph representation, wherein the annotated medical domain ontology comprises a medical ontology dataset configured to tag as an annotation the one or more medical entities along with a corresponding medical semantic type, prune a set of irrelevant information based on the annotated medical domain ontology applied to the generated semantic graph representation, and generate a medically relevant summary for the medical report based on the extracted one or more medical entities and excluding the pruned set of irrelevant information based on the annotated medical domain ontology.

According to another embodiment of the present disclosure, a computer-implemented method to implement the one or more logical processes as described herein is within the scope of the disclosure.

Accordingly to yet another embodiment, method for intelligent pipeline summarization for a medical report of a patient comprises: receiving one or more medical records associated with the patient for summarization for the medical report of the patient; extracting one or more potential headings from one or more phrases of a medical record text of the one or more medical records; comparing the one or more potential headings with one or more keyword headings of a heading seed set to generate, upon a match, one or more segmentation headings; and segmenting, via a text segmentation algorithm of an artificial intelligence model, one or more sections of the medical record text of the one or more medical records as a set of segmented texts based on the one or more segmentation headings. The method may further comprise generating a semantic graph representation based on at least the set of segmented texts that is representative of a semantic relationship as a medical semantic type between at least a plurality of nodes; extracting one or more medical entities based on an annotated medical domain ontology from the generated semantic graph representation; pruning a set of irrelevant information based on the annotated medical domain ontology applied to the generated semantic graph representation; and generating a medically relevant summary for the medical report based on the extracted one or more medical entities and excluding the pruned set of irrelevant information based on the annotated medical domain ontology.

The additional features provided by the embodiments described herein will be more fully understood in view of the following detailed description, in conjunction with the drawings. While the intelligent pipeline summary systems and methods described herein are directed to a medical industry, other industries are contemplated and within the scope of this disclosure.

### BRIEF DESCRIPTION OF THE DRAWINGS

The embodiments set forth in the drawings are illustrative and exemplary in nature and not intended to limit the subject matter defined by the claims. The following detailed description of the illustrative embodiments can be understood when read in conjunction with the following drawings, where like structure is indicated with like reference numerals in which:

FIG. 1 schematically depicts a system, according to one or more embodiments shown and described herein;

FIG. 2 depicts an illustrative flowchart of a computer-implemented method utilizing the system of FIG. 1, according to one or more embodiments shown and described herein;

FIG. 3 depicts an exemplary confusion matrix resulting from an implementation of the system of FIG. 1 and computer-implemented method of FIG. 2, according to one or more embodiments shown and described herein;

FIG. 4A depicts an exemplary chart of text segmentation on subjective information of a patient, showing segment lengths over text, using the system of FIG. 1, according to one or more embodiments shown and described herein; and

FIG. 4B depicts an exemplary histogram chart of segment lengths directed to text segmentation on subjective information of a patient using the system of FIG. 1, according to one or more embodiments shown and described herein.

### DETAILED DESCRIPTION

In various embodiments described herein, systems and computer-implemented methods of for a medical report summarization intelligent pipeline tool assist to provide more efficient and streamlined summarization of medical reports. Medical summarizers spend excessive time summarizing long, unstructured medical records with varying formats. Medical records are generally long reports conveying detailed diagnosis, test results, and findings about a patient. Each record generally contains a patient's medical history and consultation information by various facilities and providers interacting with the patient, and formatting can vary between providers. Such reports may be very long, as such as between 200-1000 pages, providing a challenge for a medical summarizer to efficiently assess and summarize the information in an efficient amount of time and avoiding errors that could negatively impact health of a patient.

Embodiments herein describe an end-to-end pipeline for summarizing such medical records. The pipeline inputs a long medical record and generates a summarized medical report. Medical ontology and semantic graph representation are incorporated by the intelligent pipeline tool to automatically generate an abstract medical summary in near or real-time in a manner not practically performed in a human mind.

Embodiments of the present disclosure are thus directed to systems and computer-implemented methods for a report summarization intelligent pipeline tool, such as a medical report, as will now be described in more detail herein with reference to the drawings and where like numbers refer to like structures. In embodiments, the report summarization intelligent pipeline tool for an industry area, such as medical records, may filter industry terms, such as clinical and/or medical ontology terms, from reports to include in the generated report summarization. The report summarization intelligent pipeline tool described herein is configured to provide an end-to-end pipeline to generate a summarization for an industry incorporating such industry terms per a domain-independent model utilized across industries with in-domain ontology (i.e., medial ontology for a medical industry, legal ontology for a legal industry, and the like). The tool may be utilized alongside other intelligent or computing tools or models, such as language models T5, PEGASUS, and BART. The tool may further utilize a pipeline to automatically generate an abstract for a medical summary in a Subjective, Objective, Assessment, and Plan (SOAP) format.

SOAP is a method of documentation used by medical practitioners to record the patient's medical progress. SOAP is a standard format that health organizations follow to reduce ambiguity when various health facilities and providers consult the patient. The Subjective component is the first component of the medical process that consists of information that the patient shares about their current symptoms, signs, and previous history. Components such as the initial evaluation of the patient, chief complaints (CC)-primary and secondary complaints, history of present illness (HPI), review of systems (ROS), and pain score levels come under subjective information. The Objective component is the information that the medical practitioner observes while assessing the patient, which may consist of medical diagnosis, lab reports, and physical assessment such as vital signs, blood pressure (BP), range of motion, palpation, muscle tenderness, and others. The Assessment component may include the patient's progress, prognosis, prescriptions, treatment modalities, and therapy options. The Plan component defines the actions taken by medical practitioners to order different labs, diagnostics, referrals, and medications. Some medical practitioners often group assessment and plan components together. Diagnostics consists of lab reports such as XRays, radiography, CT scans, MRIs, and others. The medical diagnosis may consist of impressions of medical professionals such as doctors that identify the nature of a medical problem for a patient through examination of the signs or symptoms.

In embodiments herein, the report summarization intelligent pipeline tool described herein is configured to generate a summarized industry report in a SOAP format to assist industry practitioners, such as medical practitioners, in consultations such as patient or client consultations. In medical report embodiments, from a long medical record including information directed to diagnostic reports, prescriptions, and consultations from various providers, the tool described herein is configured to automatically generate a medically relevant summary. A medical domain ontology is integrated with a generated semantic graph to prune irrelevant information and generate the medically relevant summary.

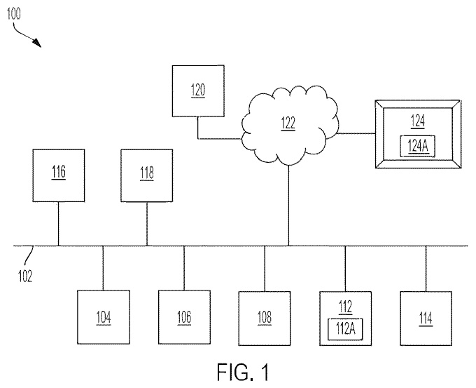

Referring now to FIG. 1, an embodiment of a system 100 as described herein includes a communication path 102, one or more processors 104, a memory component 106, an artificial intelligence module 112, a machine learning sub-module 112A of the artificial intelligence module 112, one or more databases 114, a semantic graph generation module 116, a network interface hardware 118, a network 122, a server 120, a device 124, such as a computing device, and a user interface 124 for display on the device. The various components of the system 100 and the interaction thereof will be described in detail below. In embodiments herein, the system 100 comprises a memory as the memory component 106 storing computer-executable instructions that, when executed by a processor 104, cause the system to one or more logical processes as described herein, and/or methods are configured to implement one or more logical processes as described herein (such as process 200 of FIG. 1, described in greater detail further below).

While only one server 120 and one device 124 is illustrated in FIG. 1, the system 100 can comprise multiple servers containing one or more applications and/or computing devices. In some embodiments, the system 100 is implemented using a wide area network (WAN) or network 122, such as an intranet or the internet. The device 124 may include digital systems and other devices permitting connection to and navigation of the network 122. It is contemplated and within the scope of this disclosure that the device 124 may be a personal computer, a laptop device, a smart mobile device such as a smart phone or smart pad, or the like. Other system 100 variations allowing for communication between various geographically diverse components are possible. The lines depicted in FIG. 1 indicate communication rather than physical connections between the various components.

The system 100 comprises the communication path 102. The communication path 102 may be formed from any medium that is capable of transmitting a signal such as, for example, conductive wires, conductive traces, optical waveguides, or the like, or from a combination of mediums capable of transmitting signals. The communication path 102 communicatively couples the various components of the system 100. As used herein, the term “communicatively coupled” means that coupled components are capable of exchanging data signals with one another such as, for example, electrical signals via conductive medium, electromagnetic signals via air, optical signals via optical waveguides, and the like.

The system 100 of FIG. 1 also comprises the one or more processors 104. Each processor 104 can be any device capable of executing machine-readable instructions. Accordingly, each processor 104 may be a controller, an integrated circuit, a microchip, a computer, or any other computing device. Each processor 104 is communicatively coupled to the other components of the system 100 by the communication path 102. Accordingly, the communication path 102 may communicatively couple any number of processors 104 with one another, and allow the modules coupled to the communication path 102 to operate in a distributed computing environment. Specifically, each of the modules can operate as a node that may send and/or receive data.

The illustrated system 100 further comprises the memory component 106, which is coupled to the communication path 102 and communicatively coupled to a processor 104 of the one or more processors 104. The memory component 106 may be a non-transitory computer readable medium or non-transitory computer readable memory and may be configured as a nonvolatile computer readable medium. The memory component 106 may comprise RAM, ROM, flash memories, hard drives, or any device capable of storing machine-readable instructions such that the machine-readable instructions can be accessed and executed by the processor 104. The machine readable instructions may comprise logic or algorithm(s) written in any programming language such as, for example, machine language that may be directly executed by the processor 104, or assembly language, object-oriented programming (OOP), scripting languages, microcode, etc., that may be compiled or assembled into machine readable instructions and stored on the memory component 106. Alternatively, the machine readable instructions may be written in a hardware description language (HDL), such as logic implemented via either a field-programmable gate array (FPGA) configuration or an application-specific integrated circuit (ASIC), or their equivalents. Accordingly, the methods described herein may be implemented in any conventional computer programming language, as pre-programmed hardware elements, or as a combination of hardware and software components.

Still referring to FIG. 1, as noted above, the system 100 comprises a display, such as a graphical user interface (GUI) 124A, on a screen of the device 124 for providing visual output such as, for example, information, graphical reports, messages, or a combination thereof. The display on the screen of the device 124 is coupled to the communication path 102 and communicatively coupled to the processor 104. Accordingly, the communication path 102 communicatively couples the display to other modules of the system 100. The display can comprise any medium capable of transmitting an optical output such as, for example, a cathode ray tube, light emitting diodes, a liquid crystal display, a plasma display, or the like. Additionally, it is noted that the display or the device 124 can comprise at least one of the processor 104 and the memory component 106. While the system 100 is illustrated as a single, integrated system in FIG. 1, in other embodiments, the systems can be independent systems.

The system 100 comprises the artificial intelligence module 112 configured to generate a summarization of reports, as will be described in greater detail further below. The machine-learning sub-module 112A of the artificial intelligence module 112 is configured to apply machine-learning models to the artificial intelligence models to generate the report summarizations and provide machine-learning capabilities to a neural network, as described in greater detail further below. The semantic graph generation module 116 is configured to generate a schematic graph for use with the artificial intelligence module 112 as described herein and in greater detail further below.

The artificial intelligence module 112, the machine-learning sub-module 112A, and the semantic graph generation module 116 are coupled to the communication path 102 and communicatively coupled to the processor 104. As will be described in further detail below, the processor 104 may process the input signals received from the system modules and/or extract information from such signals.

Data stored and manipulated in the system 100 as described herein is utilized by the artificial intelligence module 112, which is able to leverage a cloud computing-based network configuration such as the cloud to apply Machine Learning and Artificial Intelligence. This machine learning application may create models via the machine-learning sub-module 112A that can be applied by the system 100, to make it more efficient and intelligent in execution. As an example and not a limitation, the artificial intelligence module 112 may include artificial intelligence components selected from the group consisting of an artificial intelligence engine, Bayesian inference engine, and a decision-making engine, and may have an adaptive learning engine further comprising a deep neural network learning engine.

The system 100 further includes the network interface hardware 118 for communicatively coupling the system 100 with a computer network such as network 122. The network interface hardware 118 is coupled to the communication path 102 such that the communication path 102 communicatively couples the network interface hardware 118 to other modules of the system 100. The network interface hardware 118 can be any device capable of transmitting and/or receiving data via a wireless network. Accordingly, the network interface hardware 118 can comprise a communication transceiver for sending and/or receiving data according to any wireless communication standard. For example, the network interface hardware 118 can comprise a chipset (e.g., antenna, processors, machine readable instructions, etc.) to communicate over wired and/or wireless computer networks such as, for example, wireless fidelity (Wi-Fi), WiMax, Bluetooth, IrDA, Wireless USB, Z-Wave, ZigBee, or the like.

Still referring to FIG. 1, data from various applications running on device 124 can be provided from the device 124 to the system 100 via the network interface hardware 118. The device 124 can be any device having hardware (e.g., chipsets, processors, memory, etc.) for communicatively coupling with the network interface hardware 118 and a network 122. Specifically, the device 124 can comprise an input device having an antenna for communicating over one or more of the wireless computer networks described above.

The network 122 can comprise any wired and/or wireless network such as, for example, wide area networks, metropolitan area networks, the internet, an intranet, satellite networks, or the like. Accordingly, the network 122 can be utilized as a wireless access point by the device 124 to access one or more servers (e.g., a server 120). The server 120 and any additional servers generally comprise processors, memory, and chipset for delivering resources via the network 122. Resources can include providing, for example, processing, storage, software, and information from the server 120 to the system 100 via the network 122. Additionally, it is noted that the server 120 and any additional servers can share resources with one another over the network 122 such as, for example, via the wired portion of the network, the wireless portion of the network, or combinations thereof. Where used herein, “a first element, a second element, or combinations thereof” reference an “and/or” combination similar to use herein of “at least one of a first element or a second element.”

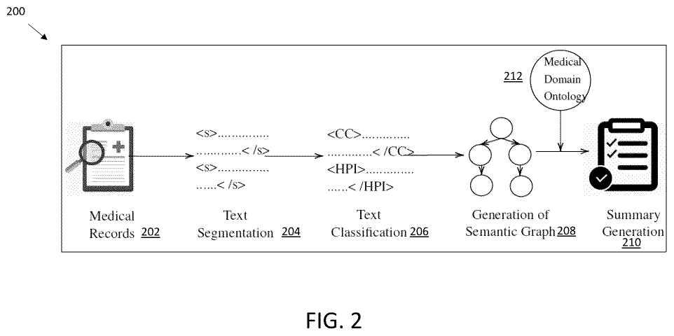

Referring to FIG. 2, an example process 200 of a computer-implemented method using the system 100 of FIG. 1 to summary a report such as a medical report 202 is depicted. For an intelligent pipeline summarization for a medical report of a patient of the process 200, one or more medical records such as one or more medical reports 202 associated with the patient are received for summarization for the corresponding medical report of the patient.

A pipeline for the report summarization intelligent pipeline tool as described herein may include the stages shown in the process 200 of FIG. 200, such as text segmentation 204, text classification 206, semantic graph representation 208, and summary generation 210. A medical domain ontology 212 is integrated with the generated semantic graph representation 210 to prune irrelevant information and generate the medically relevant summary.

In embodiments, industry summarizers with relevant backgrounds may participate in an annotation process that may be validated prior to use of the annotations to a dataset for the report summarization intelligent pipeline tool. In an embodiment for a medical report summarization, medical summarizers may annotate two datasets such as a classification dataset and a medical ontology dataset. The classification dataset may classify the texts of the reports into a number of predefined categories, such as ROS, CC, HPI, and the like. In an embodiment, the number of predefined categories may be 28 predefined categories. The medical ontology dataset may tag the medical entities along with their semantic types such as disease, symptom, medical and the like. The medical semantic type may be disease, symptom, medical or combinations thereof, and the annotated medical domain ontology may include a medical ontology dataset configured to tag as an annotation the one or more medical entities along with a corresponding medical semantic type.

Scores such as Fleiss' kappa and Krippendorff's Alpha models may be tested and used to measure an annotation task. Fleiss' kappa measures how similar two annotators annotate up to an agreement for text classification. Krippendorff's Alpha measures inter-rater variability, indicating inter-annotator agreement on the medical entity ontology task. One testing of the report summarization intelligent pipeline tool as described herein and as presented herein involves five medical summarizers with relevant backgrounds to participate in an annotation process. In a test of the five medical annotators, the medical annotates scored 0.91 for Fleiss' kappa 155 and 0.82 for Krippendorff's Alpha. In the test, 30, 156 medical records 202 were provided as input, and 157 texts were segmented 204. The dataset was split in train, valid, and test modes with 582, 73, and 73 samples, respectively.

With respect to text segmentation 204 of FIG. 2, input medical reports 202 included unstructured documents mainly consisting of SOAP format, where each medical provider could follow a different template for medical reports. In embodiments, the medically relevant summary is automatically generated in a Subjective, Objective, Assessment, and Plan (SOAP) format, and at least a portion of the one or more medical records is in a format different from the SOAP format. The algorithm for text segmentation 204 includes at least the following steps: pre-processing, heading detection, and segmentation iteratively.

In the pre-processing phase, data cleaning and normalization techniques are incorporated. One or more potential headings may be extracted from one or more phrases of a medical record text of the one or more medical records. The one or more potential headings may be extracted from the one or more phrases of the medical record text of the one or more medical records when the one or more phrases comprise a word count threshold of less than four words. In an embodiment, only those phrases whose word count threshold is less than four are extracted as the potential headings from running text.

The one or more potential headings may be compared with one or more keyword headings of a heading seed set to generate, upon a match, one or more segmentation headings. In the heading detection phase, annotations generated by the medical summarizers are taken as keyword headings documented for SOAP formatting and used as an initial seed set for cue phrases matching the potential headings from the pre-processing step. Upon a successful match, the match may be used as a heading for segmentation.

In the segmentation phase, segmentation is performed after identification of headings in the heading detection phase. Via a text segmentation algorithm of an artificial intelligence model, one or more sections of the medical record text of the one or more medical records may be segmented as a set of segmented texts based on the one or more segmentation headings. From the segmented sections, more potential headings may be identified using the pre-processing phase as step one from the initial set of medical documents 202. An additional potential heading may be identified from the set of segmented texts based on the one or more segmentation headings. A record of these new potential headings count from iterated step one may be kept. If the new potential heading count in many documents crosses the utilized threshold and contains a similar phrase to previous headings, the new heading may be added to the seed set. In an embodiment, the additional potential heading may be added to the heading seed set when the one or more phrases of the additional potential heading comprise a word count threshold of less than four words and the additional potential heading comprises at least one similar phrase when compared to a segmentation heading of the one or segmentation headings. Thus, the iterative phases aid to expand seed headings and identify new headings. Even without an initial list of headings in the medical report, SOAP sections may be identified respectively via these iterative phases.

Via a text classification algorithm of the artificial intelligence model, the set of segmented texts may be classified with one or more pre-defined medical tags to generate one or more groups of classified text, and a semantic graph representation (e.g., semantic graph 208) may be generated based on the one or more groups of classified text. The number of predefined categories may include subjective information, objective information, assessment information, plan information, or combinations thereof, and the medically relevant summary for the medical report may be generated in a subjective, objective, assessment, and plan (SOAP) information format as set forth above. The subjective information may include one or more chief complaints (CC), a history of a present illness (HPI), a review of systems (ROS), a pain score level, or combinations thereof. The objective information may include observation information by a medical practitioner comprising at least one of medical diagnosis, lab reports, vital signs, blood pressure (BP), range of motion, palpation, muscle tenderness, or combinations thereof. The assessment information may include progress of the patient, prognosis, one or more prescriptions, one or more treatment modalities, one or more therapy options, or combinations thereof. The plan information may include one or more actions by a medical practitioner comprising one or more lab orders, diagnostics orders, one or more referrals, and one or more medication orders.

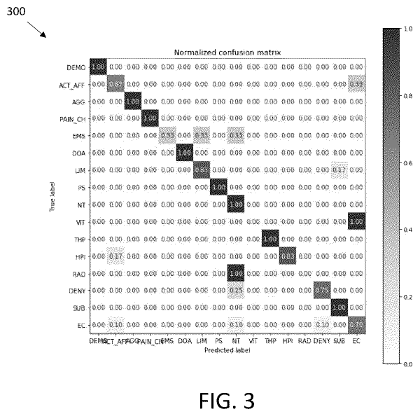

With respect to text classification 206 of FIG. 2, the segmented texts are classified with medical tags in this phase. In an embodiment described herein and above, the segmented texts were annotated into 28 categories to extract the content in SOAP format. To achieve this, a Bio-Clinical-BERT model as a pre-trained model was fine-tuned on a tagged dataset, which Bio-Clinical-BERT model was trained on a MIMIC III database. MIMIC III is a dataset that contains ICU patients' medical notes and electronic health records. Hence, the model is familiar with medical terms like HPIs, labs, complaints, and the like. The model was trained for 30 epochs, with a learning rate of 2e-5 and weight decay of 0.01. Early stopping criteria was used with three callbacks to avoid over-fitting with evaluation at every 50 steps. The annotated dataset was class imbalanced, so the metric for the best model was selected as the F1 score. The model gave a 0.77 validation F1-score. FIG. 3 depicts an exemplary confusion matrix 300 resulting from the test implementation of the system of FIG. 1 and computer-implemented method of FIG. 2 as described above with respect to text classification 206.

Via a semantic graph generation module 116 (FIG. 1), a semantic graph representation as semantic graph 208 may be generated based on at least the set of segmented texts that is representative of a semantic relationship as a medical semantic type between at least a plurality of nodes. With respect to the generation of semantic graph 208 stage of FIG. 2, a semantic graph is a network type representative of a semantic relationship between nodes. The nodes are concepts, and the edges are indicative of sematic relationships therebetween. For example, in a medical domain, pneumonia may be node 1 and may be connected to lungs as node 2 with a semantic type of disease. AMR may be used to generate the semantic graph 208.

AMR is a semantic representation representing sentences in rooted, labeled, directed acyclic graphs (DAG). In a generation embodiment, amrlib2 is used to generate and visualize the AMR representation of extracted SOAP content. A parse_xfm_bart_base model is used for converting the extracted content into AMR representation. Internally, the model uses BART_{base} model available at a Hugging Face repository. An objective is to generate a medical summary without any unrelated information. The annotated medical domain ontology may be used to extract the medical entities from the semantic graph 208.

In embodiments, one or more medical entities may be extracted based on an annotated medical domain ontology from the generated semantic graph representation (e.g., the semantic graph 208). A set of irrelevant information may be pruned based on the annotated medical domain ontology (e.g., the medical domain ontology 212) applied to the generated semantic graph representation. A medically relevant summary for the medical report may be generated as a summary generation 210 based on the extracted one or more medical entities and excluding the pruned set of irrelevant information based on the annotated medical domain ontology.

With respect to the medical domain ontology 212 stage of FIG. 2, the medical domain ontology 212 may be integrated into the generated semantic graph 208. When generated, the generated semantic graph 208 may include irrelevant information such as vehicle details in motor vehicle accident incidents, patent work details, and the like. An integration of the medical domain ontology 212 into the generated semantic graph 208 permits a pruning of such irrelevant information from the semantic graph 208.

The integration may occur via applying entity chunking to obtain valid unigrams, bigrams, and trigrams. Chunks following tag patterns NOUN-NOUN and ADJ-NOUN may be retained for retrieving all of the potential entities. A technique such as a simstring technique may be used as an algorithm for approximate string matching. Such a technique may support many similarity measures for string matching, such as cosine, Jaccard, word n-gram, and character n-gram. A dictionary database may be initialized, and all entities from the domain entity list may be added to a simstring database for fast matching. A character n-gram feature and cosine measure may be used to check the similarity. Apart from the extracted medical entities, all other remaining entities may then be pruned from the semantic graph along with their connected relations. The final medical semantic graph 208 as pruned is then passed as input to the next phase of summary generation 210.

With respect to the summary generation 210 stage of FIG. 2, the stage is a final pipeline stage and involves summary generation 210 from the pruned semantic graph 208. A T5 model may be used to generate the summary for summary generation 210 from the pruned semantic graph 208 containing the medical entities.

An embodiment of the resulting summary generation 210 was evaluated. In particular, the models were evaluated on the test set using standard metrics. ROUGE and BERTscore were used to automatically evaluate the generated summaries against target summaries annotated by medical summarizers. ROUGE (Recall-Oriented Understudy for Gisting Evaluation) is an evaluation metric used widely in automatic summarization and machine translation tasks. ROUGE compares the generated summary against the set of references produced by humans and is case insensitive. ROUGE computes the scores based on overlap of unigram (ROUGE-1), bigram (ROUGE-2), and Longest Common Subsequence (LCS) (ROUGE-L and ROUGE-LSum). BERTScore computes similarity score between each token of generated and reference summary. BERTScore uses BERT contextual embeddings for token similarity.

In an evaluation embodiment, to evaluate the quality of the generated summary from the five annotating medical summarizers, 10 generated summaries were randomly selected from each model and the summaries analyzed with the assistance of three summarizers other than the ones who annotated the dataset. Three metrics were employed as fluency, adequacy, and entity relevance for assessing the accuracy of the model generations. All the metrics scale from 1 to 5, with a higher number indicating a more positive, better scoring.

Fluency—the fluency metric measures the grammar of the sentence i.e., to analyze that the text is easy to understand and does not miss any fragments. The ratings are based on whether the summary is incoherent, dis-fluent, non-native, satisfactory, and correct English, respectively.

Adequacy—the adequacy metric measures whether the generated summary is meaningful and shows relevancy to the input article. The ratings are based on whether the summary is none, little meaning, much meaning, most meaning, and all meaning, respectively.

Entity relevance—the entity metric measures whether the summary contains factually correct medical entities mentioned in the input article. The ratings are higher and better if the summary contains all entities correctly. As these scores are averaged based on ratings received from the three summarizers, a Fleiss' kappa score was also computed to assess the inter-annotator agreement (i.e., agreement between the annotators).

BART and T5 were used as the evaluation baseline models. BART consists of bidirectional encoder (like BERT) and a left-to-right decoder (like GPT). BART pre-training is done with shuffling of the order of sentences and masking of tokens. The pre-trained BART model fine-tuned on a XSUM dataset was used for summarization.

T5 is a sequence-to-sequence model that is pre-trained on multiple self-supervised and supervised tasks. T5 also uses corrupted tokens in the self-supervised training. The T5 base model fine-tuned on the XSUM dataset was used for summarization.

The F1-score of both the metrics was computed for the evaluation. The results using automatic evaluation metrics is shown in Table 1.

TABLE 1
ROUGE and BERT Scores on test data

| Method | Rouge-1 | Rouge-2 | Rouge-L | Rouge-LSum | BERTScore |
|---|---|---|---|---|---|
| BART | 0.13741 | 0.03716 | 0.11357 | 0.11360 | 0.84828 |
| T5 | 0.18235 | 0.02944 | 0.12703 | 0.12547 | 0.83238 |
| Proposed | 0.24926 | 0.11354 | 0.20931 | 0.21688 | 0.85179 |

The model described as the tool herein outperformed the baseline models on both the metrics demonstrating the efficiency of using domain ontology with the semantic graphs. When compared to the T5 baseline, the tool model described herein yields significant performance of 36.7%, 285.5% in ROUGE-1 and ROUGE-2 F1 score respectively. BART yielded slightly better BERTScore than T5. However, the tool model described herein showed a best BERTScore performance.

Table 2 shows the results of human evaluation as described herein. The tool model described herein outperformed in all the three metrics of fluency, adequacy, and entity-relevance. The tool model described herein shows consistency with the results of automatic evaluation. The kappa values are also greater than 0.75 that indicates all the annotators agree with each other.

TABLE 2
Human assessment scores for baseline and proposed model

| Method | Fluency | Adequacy | Ently Relevance | Kappa |
|---|---|---|---|---|
| BART | 3 | 2 | 1.5 | 0.87 |
| T5 | 3.2 | 2.5 | 2 | 0.86 |
| Ours | 3.2 | 3.5 | 3.5 | 0.87 |

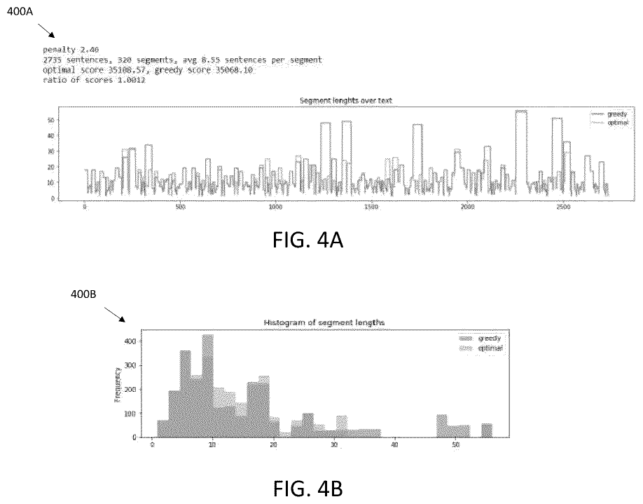

FIGS. 4A-4B show the result of text segmentation on subjective information of a patient. FIG. 4A depicts an exemplary chart of text segmentation on subjective information of a patient, showing segment lengths over text, arising from the test using the system of FIG. 1 (i.e., the tool model described herein). FIG. 4B depicts an exemplary histogram chart of segment lengths directed to text segmentation on subjective information of a patient arising from the test using the system of FIG. 1 (i.e., the tool model described herein). As observed from the histogram of FIG. 4B, an optimal result is neared using the text segmentation model.

Table 3 shows the summary generated from the models based on one input medical report. The medical entities are marked in bold.

TABLE 3
Comparison of baseline and proposed medical summary

| | |
|---|---|
| INPUT | This is a pleasant 40-year-old righ-handed female who was in her usual state of health |
| MEDICAL | when she was involved in a road accident at the beginning of March 2023. She was the |
| REPORT | restrained driver of a BMW E series that was rear ended in traffic by the at-fault driver |
| | of an 18-wheeler. As a result, her vehicle sustained moderate damage and she sustained |
| | the above injuries. She has been under treatment at City Hospital and has been attemped |
| | to be managed with medication. Unfortunately, her symptoms continued and she was |
| | experiencing referred symptoms into her right lower extremity downs to the level of mid |
| | thigh. Due to these continued radicular complaints, she was referred to this clinic for |
| | further evaluation and treatment. 2. follow up Patient presents to clinic for Lumbar PRP |
| | follow up. Patient states the injection helped relieve 65% of her pain. On a pain scale of |
| | 0 to 10, patient rates their pain on a bad day at a 9 and 4 on a good day. |
| TARGET | Road accidents in beginning of March 2023. Was restrained driver, struck by at-fault |
| SUMMARY | driver. Vehicle get Moderate damage and she sustained above injuries. Under Tx of |
| | City Hospital and has been attempted to be managed [symbol] meds. Unfortunately, her Sx |
| | cont and she was experiencing referred Sx in her Rt.LE down to level of mid thigh. |
| | Due to these cont radicular Sx, referred here for further mgmt. Here for Lumbar PRP [symbol] |
| | inj helped 65% of her pain. Pain Scale worst 9/10. Best 4/10. |
| OURS | Her symptoms continued to be pleasant when she was involved in a road accident |
| | beginning in March 2023. She experienced symptoms that were referred to the clinic |
| | for evaluation and treatment because she had continued radicular complaints. She was |
| | treated with pain scores of 9/10 and good pain score. |
| BART | A woman who was involed in a road accident in March 2023 has been referred to this |
| | clinic for further evaluation and treatment. |
| T5 | 40-year-old right-handed woman was involved in a road accident in the beginning of |
| | March 2023. she was the restrained driver of a BMW E series that was |
| | rear-ended in traffic by the at-fault driver of an 18-wheeler. |

[symbol]

indicates data missing or illegible when filed

An end-to-end automated and intelligent tool pipeline is described herein for industry summarization such as medical summarization using semantic graphs and annotated industry domain ontology (i.e., medical domain ontology). The medical summary may be automatically generated in SOAP format, a format commonly used by the medical practitioners for writing medical reports.

It is also noted that recitations herein of “at least one” component, element, etc., should not be used to create an inference that the alternative use of the articles “a” or “an” should be limited to a single component, element, etc.

It is noted that recitations herein of a component of the present disclosure being “configured” or “programmed” in a particular way, to embody a particular property, or to function in a particular manner, are structural recitations, as opposed to recitations of intended use.

Having described the subject matter of the present disclosure in detail and by reference to specific embodiments thereof, it is noted that the various details disclosed herein should not be taken to imply that these details relate to elements that are essential components of the various embodiments described herein, even in cases where a particular element is illustrated in each of the drawings that accompany the present description. Further, it will be apparent that modifications and variations are possible without departing from the scope of the present disclosure, including, but not limited to, embodiments defined in the appended claims. More specifically, although some aspects of the present disclosure are identified herein as preferred or particularly advantageous, it is contemplated that the present disclosure is not necessarily limited to these aspects.

It is noted that one or more of the following claims utilize the term “wherein” as a transitional phrase. For the purposes of defining the present disclosure, it is noted that this term is introduced in the claims as an open-ended transitional phrase that is used to introduce a recitation of a series of characteristics of the structure and should be interpreted in like manner as the more commonly used open-ended preamble term “comprising.”

### Aspects Listing

Aspect 1. A system for intelligent pipeline summarization for a medical report of a patient, the system comprising a processor and a memory storing computer-executable instructions that, when executed by the processor, cause the system to: receive one or more medical records associated with the patient for summarization for the medical report of the patient, and extract one or more potential headings from one or more phrases of a medical record text of the one or more medical records. The computer-executable instructions, when executed by the processor, further cause the system to: compare the one or more potential headings with one or more keyword headings of a heading seed set to generate, upon a match, one or more segmentation headings; segment, via a text segmentation algorithm of an artificial intelligence model, one or more sections of the medical record text of the one or more medical records as a set of segmented texts based on the one or more segmentation headings, generate a semantic graph representation based on at least the set of segmented texts that is representative of a semantic relationship as a medical semantic type between at least a plurality of nodes, extract one or more medical entities based on an annotated medical domain ontology from the generated semantic graph representation, prune a set of irrelevant information based on the annotated medical domain ontology applied to the generated semantic graph representation, and generate a medically relevant summary for the medical report based on the extracted one or more medical entities and excluding the pruned set of irrelevant information based on the annotated medical domain ontology.

Aspect 2. The system of Aspect 1, wherein the computer-executable instructions, when executed by the processor, further cause the system to: classify, via a text classification algorithm of the artificial intelligence model, the set of segmented texts with one or more pre-defined medical tags to generate one or more groups of classified text; and generate the semantic graph representation based on the one or more groups of classified text.

Aspect 3. The system of Aspect 2, wherein the one or more groups of classified text comprise a number of predefined categories comprising subjective information, objective information, assessment information, plan information, or combinations thereof, and wherein the medically relevant summary for the medical report is generated in a subjective, objective, assessment, and plan (SOAP) information format.

Aspect 4. The system of Aspect 3, wherein the subjective information comprises one or more chief complaints (CC), a history of a present illness (HPI), a review of systems (ROS), a pain score level, or combinations thereof.

Aspect 5. The system of Aspect 3 or Aspect 4, wherein the objective information comprises observation information by a medical practitioner comprising at least one of medical diagnosis, lab reports, vital signs, blood pressure (BP), range of motion, palpation, muscle tenderness, or combinations thereof.

Aspect 6. The system of any of Aspect 3 to Aspect 5, wherein the assessment information comprises progress of the patient, prognosis, one or more prescriptions, one or more treatment modalities, one or more therapy options, or combinations thereof.

Aspect 7. The system of any of Aspect 3 to Aspect 6, wherein the plan information comprises one or more actions by a medical practitioner comprising one or more lab orders, diagnostics orders, one or more referrals, and one or more medication orders.

Aspect 8. The system of any of Aspect 1 to Aspect 7, wherein the computer-executable instructions, when executed by the processor, further cause the system to: extract the one or more potential headings from the one or more phrases of the medical record text of the one or more medical records when the one or more phrases comprise a word count threshold of less than four words.

Aspect 9. The system of any of Aspect 1 to Aspect 8, wherein the computer-executable instructions, when executed by the processor, further cause the system to: identify an additional potential heading from the set of segmented texts based on the one or more segmentation headings; and add the additional potential heading to the heading seed set when the one or more phrases of the additional potential heading comprise a word count threshold of less than four words and the additional potential heading comprises at least one similar phrase when compared to a segmentation heading of the one or segmentation headings.

Aspect 10. The system of any of Aspect 1 to Aspect 9, wherein the medically relevant summary is automatically generated in a Subjective, Objective, Assessment, and Plan (SOAP) format, wherein at least a portion of the one or more medical records is in a format different from the SOAP format.

Aspect 11. The system of any of Aspect 1 to Aspect 10, wherein the medical semantic type comprises disease, symptom, medical or combinations thereof, and wherein the annotated medical domain ontology comprises a medical ontology dataset configured to tag as an annotation the one or more medical entities along with a corresponding medical semantic type.

Aspect 12. A system for intelligent pipeline summarization for a medical report of a patient, the system comprising a processor and a memory storing computer-executable instructions. When executed by the processor, the computer-executable instructions cause the system to receive one or more medical records associated with the patient for summarization for the medical report of the patient, and extract one or more potential headings from one or more phrases of a medical record text of the one or more medical records. The computer-executable instructions, when executed by the processor, further cause the system to: compare the one or more potential headings with one or more keyword headings of a heading seed set to generate, upon a match, one or more segmentation headings, segment, via a text segmentation algorithm of an artificial intelligence model, one or more sections of the medical record text of the one or more medical records as a set of segmented texts based on the one or more segmentation headings, and classify, via a text classification algorithm of the artificial intelligence model, the set of segmented texts with one or more pre-defined medical tags to generate one or more groups of classified text, wherein the one or more groups of classified text comprise a number of predefined categories. The computer-executable instructions, when executed by the processor, further cause the system to: generate a semantic graph representation based on at least the one or more groups of classified text that is representative of a semantic relationship as a medical semantic type between at least a plurality of nodes, extract one or more medical entities based on an annotated medical domain ontology from the generated semantic graph representation, wherein the annotated medical domain ontology comprises a medical ontology dataset configured to tag as an annotation the one or more medical entities along with a corresponding medical semantic type, prune a set of irrelevant information based on the annotated medical domain ontology applied to the generated semantic graph representation, and generate a medically relevant summary for the medical report based on the extracted one or more medical entities and excluding the pruned set of irrelevant information based on the annotated medical domain ontology.

Aspect 13. The system of Aspect 12, wherein the number of predefined categories comprises subjective information, objective information, assessment information, plan information, or combinations thereof, and wherein the medically relevant summary for the medical report is generated in a subjective, objective, assessment, and plan (SOAP) information format.

Aspect 14. The system of Aspect 13, wherein (i) the subjective information comprises one or more chief complaints (CC), a history of a present illness (HPI), a review of systems (ROS), a pain score level, or combinations thereof, (ii) the objective information comprises observation information by a medical practitioner comprising at least one of medical diagnosis, lab reports, vital signs, blood pressure (BP), range of motion, palpation, muscle tenderness, or combinations thereof, (iii) the assessment information comprises progress of the patient, prognosis, one or more prescriptions, one or more treatment modalities, one or more therapy options, or combinations thereof, and (iv) the plan information comprises one or more actions by a medical practitioner comprising one or more lab orders, diagnostics orders, one or more referrals, and one or more medication orders.

Aspect 15. The system of any of Aspect 12 to Aspect 14, wherein the computer-executable instructions, when executed by the processor, further cause the system to: extract the one or more potential headings from the one or more phrases of the medical record text of the one or more medical records when the one or more phrases comprise a word count threshold of less than four words.

Aspect 16. The system of any of Aspect 12 to Aspect 15, wherein the computer-executable instructions, when executed by the processor, further cause the system to: identify an additional potential heading from the set of segmented texts based on the one or more segmentation headings; and add the additional potential heading to the heading seed set when the one or more phrases of the additional potential heading comprise a word count threshold of less than four words and the additional potential heading comprises at least one similar phrase when compared to a segmentation heading of the one or segmentation headings.

Aspect 17. The system of any of Aspect 12 to Aspect 16, wherein the medically relevant summary is automatically generated in a Subjective, Objective, Assessment, and Plan (SOAP) format, wherein at least a portion of the one or more medical records is in a format different from the SOAP format.

Aspect 18. The system of any of Aspect 12 to Aspect 17, wherein the medical semantic type comprises disease, symptom, medical or combinations thereof.

Aspect 19. A method for intelligent pipeline summarization for a medical report of a patient, the method comprising: receiving one or more medical records associated with the patient for summarization for the medical report of the patient; extracting one or more potential headings from one or more phrases of a medical record text of the one or more medical records; comparing the one or more potential headings with one or more keyword headings of a heading seed set to generate, upon a match, one or more segmentation headings; and segmenting, via a text segmentation algorithm of an artificial intelligence model, one or more sections of the medical record text of the one or more medical records as a set of segmented texts based on the one or more segmentation headings. The method may further comprise generating a semantic graph representation based on at least the set of segmented texts that is representative of a semantic relationship as a medical semantic type between at least a plurality of nodes; extracting one or more medical entities based on an annotated medical domain ontology from the generated semantic graph representation; pruning a set of irrelevant information based on the annotated medical domain ontology applied to the generated semantic graph representation; and generating a medically relevant summary for the medical report based on the extracted one or more medical entities and excluding the pruned set of irrelevant information based on the annotated medical domain ontology.

Aspect 20. The method of Aspect 19, further comprising classifying, via a text classification algorithm of the artificial intelligence model, the set of segmented texts with one or more pre-defined medical tags to generate one or more groups of classified text; and generating, via a semantic graph generation module, the semantic graph representation based on the one or more groups of classified text.

What is claimed is:

1. A system for intelligent pipeline summarization for a medical report of a patient, the system comprising:
a processor; and
a memory storing computer-executable instructions that, when executed by the processor, cause the system to:
receive one or more medical records associated with the patient for summarization for the medical report of the patient;
extract one or more potential headings from one or more phrases of a medical record text of the one or more medical records;
compare the one or more potential headings with one or more keyword headings of a heading seed set to generate, upon a match, one or more segmentation headings;
segment, via a text segmentation algorithm of an artificial intelligence model, one or more sections of the medical record text of the one or more medical records as a set of segmented texts based on the one or more segmentation headings;
generate a semantic graph representation based on at least the set of segmented texts that is representative of a semantic relationship as a medical semantic type between at least a plurality of nodes;
extract one or more medical entities based on an annotated medical domain ontology from the generated semantic graph representation;
prune a set of irrelevant information based on the annotated medical domain ontology applied to the generated semantic graph representation; and
generate a medically relevant summary for the medical report based on the extracted one or more medical entities and excluding the pruned set of irrelevant information based on the annotated medical domain ontology.

2. The system of claim 1, wherein the computer-executable instructions, when executed by the processor, further cause the system to:
classify, via a text classification algorithm of the artificial intelligence model, the set of segmented texts with one or more pre-defined medical tags to generate one or more groups of classified text; and
generate the semantic graph representation based on the one or more groups of classified text.

3. The system of claim 2, wherein the one or more groups of classified text comprise a number of predefined categories comprising subjective information, objective information, assessment information, plan information, or combinations thereof, and wherein the medically relevant summary for the medical report is generated in a subjective, objective, assessment, and plan (SOAP) information format.

4. The system of claim 3, wherein the subjective information comprises one or more chief complaints (CC), a history of a present illness (HPI), a review of systems (ROS), a pain score level, or combinations thereof.

5. The system of claim 3, wherein the objective information comprises observation information by a medical practitioner comprising at least one of medical diagnosis, lab reports, vital signs, blood pressure (BP), range of motion, palpation, muscle tenderness, or combinations thereof.

6. The system of claim 3, wherein the assessment information comprises progress of the patient, prognosis, one or more prescriptions, one or more treatment modalities, one or more therapy options, or combinations thereof.

7. The system of claim 3, wherein the plan information comprises one or more actions by a medical practitioner comprising one or more lab orders, diagnostics orders, one or more referrals, and one or more medication orders.

8. The system of claim 1, wherein the computer-executable instructions, when executed by the processor, further cause the system to:
extract the one or more potential headings from the one or more phrases of the medical record text of the one or more medical records when the one or more phrases comprise a word count threshold of less than four words.

9. The system of claim 1, wherein the computer-executable instructions, when executed by the processor, further cause the system to:
identify an additional potential heading from the set of segmented texts based on the one or more segmentation headings; and
add the additional potential heading to the heading seed set when the one or more phrases of the additional potential heading comprise a word count threshold of less than four words and the additional potential heading comprises at least one similar phrase when compared to a segmentation heading of the one or segmentation headings.

10. The system of claim 1, wherein the medically relevant summary is automatically generated in a Subjective, Objective, Assessment, and Plan (SOAP) format, wherein at least a portion of the one or more medical records is in a format different from the SOAP format.

11. The system of claim 1, wherein the medical semantic type comprises disease, symptom, medical or combinations thereof, and wherein the annotated medical domain ontology comprises a medical ontology dataset configured to tag as an annotation the one or more medical entities along with a corresponding medical semantic type.

12. A system for intelligent pipeline summarization for a medical report of a patient, the system comprising:
a processor; and
a memory storing computer-executable instructions that, when executed by the processor, cause the system to:
receive one or more medical records associated with the patient for summarization for the medical report of the patient;
extract one or more potential headings from one or more phrases of a medical record text of the one or more medical records;
compare the one or more potential headings with one or more keyword headings of a heading seed set to generate, upon a match, one or more segmentation headings;
segment, via a text segmentation algorithm of an artificial intelligence model, one or more sections of the medical record text of the one or more medical records as a set of segmented texts based on the one or more segmentation headings;
classify, via a text classification algorithm of the artificial intelligence model, the set of segmented texts with one or more pre-defined medical tags to generate one or more groups of classified text, wherein the one or more groups of classified text comprise a number of predefined categories;
generate a semantic graph representation based on at least the one or more groups of classified text that is representative of a semantic relationship as a medical semantic type between at least a plurality of nodes;
extract one or more medical entities based on an annotated medical domain ontology from the generated semantic graph representation, wherein the annotated medical domain ontology comprises a medical ontology dataset configured to tag as an annotation the one or more medical entities along with a corresponding medical semantic type;
prune a set of irrelevant information based on the annotated medical domain ontology applied to the generated semantic graph representation; and
generate a medically relevant summary for the medical report based on the extracted one or more medical entities and excluding the pruned set of irrelevant information based on the annotated medical domain ontology.

13. The system of claim 12, wherein the number of predefined categories comprises subjective information, objective information, assessment information, plan information, or combinations thereof, and wherein the medically relevant summary for the medical report is generated in a subjective, objective, assessment, and plan (SOAP) information format.

14. The system of claim 13, wherein (i) the subjective information comprises one or more chief complaints (CC), a history of a present illness (HPI), a review of systems (ROS), a pain score level, or combinations thereof, (ii) the objective information comprises observation information by a medical practitioner comprising at least one of medical diagnosis, lab reports, vital signs, blood pressure (BP), range of motion, palpation, muscle tenderness, or combinations thereof, (iii) the assessment information comprises progress of the patient, prognosis, one or more prescriptions, one or more treatment modalities, one or more therapy options, or combinations thereof, and (iv) the plan information comprises one or more actions by a medical practitioner comprising one or more lab orders, diagnostics orders, one or more referrals, and one or more medication orders.

15. The system of claim 12, wherein the computer-executable instructions, when executed by the processor, further cause the system to:
extract the one or more potential headings from the one or more phrases of the medical record text of the one or more medical records when the one or more phrases comprise a word count threshold of less than four words.

16. The system of claim 12, wherein the computer-executable instructions, when executed by the processor, further cause the system to:
identify an additional potential heading from the set of segmented texts based on the one or more segmentation headings; and
add the additional potential heading to the heading seed set when the one or more phrases of the additional potential heading comprise a word count threshold of less than four words and the additional potential heading comprises at least one similar phrase when compared to a segmentation heading of the one or segmentation headings.

17. The system of claim 12, wherein the medically relevant summary is automatically generated in a Subjective, Objective, Assessment, and Plan (SOAP) format, wherein at least a portion of the one or more medical records is in a format different from the SOAP format.

18. The system of claim 1, wherein the medical semantic type comprises disease, symptom, medical or combinations thereof.

19. A method for intelligent pipeline summarization for a medical report of a patient, the method comprising:
receiving one or more medical records associated with the patient for summarization for the medical report of the patient;
extracting one or more potential headings from one or more phrases of a medical record text of the one or more medical records;
comparing the one or more potential headings with one or more keyword headings of a heading seed set to generate, upon a match, one or more segmentation headings;
segmenting, via a text segmentation algorithm of an artificial intelligence model, one or more sections of the medical record text of the one or more medical records as a set of segmented texts based on the one or more segmentation headings;
generating a semantic graph representation based on at least the set of segmented texts that is representative of a semantic relationship as a medical semantic type between at least a plurality of nodes;
extracting one or more medical entities based on an annotated medical domain ontology from the generated semantic graph representation;
pruning a set of irrelevant information based on the annotated medical domain ontology applied to the generated semantic graph representation; and
generating a medically relevant summary for the medical report based on the extracted one or more medical entities and excluding the pruned set of irrelevant information based on the annotated medical domain ontology.

20. The method of claim 19, further comprising:
classifying, via a text classification algorithm of the artificial intelligence model, the set of segmented texts with one or more pre-defined medical tags to generate one or more groups of classified text; and
generating, via a semantic graph generation module, the semantic graph representation based on the one or more groups of classified text.
